(19) United States
(12) Patent Application Publication
(10) Pub. No.: US 2018/0103927 A1
(43) Pub. Date: Apr. 19, 2018

(54) HEADSET AND DEVICE INCLUDING A COVER

(71) Applicant: Neural Analytics, Inc., Los Angeles, CA (US)

(72) Inventors: Raymond Chung, Los Angeles, CA (US); Michael Costa, Los Angeles, CA (US); Trevor Dunlop, Los Angeles, CA (US); Roman Flores, II, Los Angeles, CA (US); Robert Hamilton, Los Angeles, CA (US); Matthew Hutter, Los Angeles, CA (US); Michael O'Brien, Los Angeles, CA (US); Leo Petrossian, Los Angeles, CA (US); Shankar Radhakrishnan, Los Angeles, CA (US); Mina Ranjbaran, Los Angeles, CA (US); Lane Stith, Los Angeles, CA (US); Matthew Sylvester, Los Angeles, CA (US); Corey Thibeault, Los Angeles, CA (US); Seth J. Wilk, Los Angeles, CA (US); Jay Yonemoto, Los Angeles, CA (US); Jan Zwierstra, Los Angeles, CA (US)

(21) Appl. No.: 15/785,348

(22) Filed: Oct. 16, 2017

Related U.S. Application Data
(60) Provisional application No. 62/409,060, filed on Oct. 17, 2016.
(60) Provisional application No. 62/412,755, filed on Oct. 25, 2016.

(51) Int. Cl. A61B 8/00; A61B 8/08

(52) U.S. Cl. CPC A61B 8/4209; A61B 8/0808; A61B 8/488

(57) ABSTRACT

A headset for placing on a head of a subject includes a device and a cover over the device. At least a portion of the cover is made from a see-through material such that a portion of the head of the subject is viewable through the cover.

### CROSS-REFERENCE TO RELATED PATENT APPLICATIONS

This application claims the benefit of and priority to U.S. provisional patent application Ser. No. 62/409,060, filed Oct. 17, 2016, the contents of which are incorporated herein by reference in its entirety. This application claims the benefit of and priority to U.S. provisional patent application Ser. No. 62/412,755, filed Oct. 25, 2016, the contents of which are incorporated herein by reference in its entirety.

### BACKGROUND

For devices that utilize a headset whose performance is optimized by remaining stable with respect to a user's head (e.g., optical devices, surgical devices, scanning devices, medical diagnostic devices, automated Transcranial Doppler devices, and so on), initial registration or alignment of the device with respect to particular areas of a patient's head is important for accurate readings by the device during operation. However, it may be difficult to determine proper initial positioning of the device due to visual impediments between an operator and the subject's head (e.g., the headset or the device itself may be blocking the view of the operator). As such, improper placement often occurs as a result.

### SUMMARY

According to various embodiments, there is provided a headset attached with a device that allows an operator to visually inspect positioning of the device with respect to a subject's head. Furthermore, according to various embodiments, the headset and/or device includes indicators that allow the operator to precisely register the device with respect to the subject's head.

According to various embodiments, there is provided a headset for placing on a head of a subject. The headset includes a device and a cover over the device. At least a portion of the cover is made from a see-through material such that a portion of the head of the subject is viewable through the cover.

In some embodiments, the device includes robotics and a probe attached to the robotics.

In some embodiments, the cover includes an aperture and the probe protrudes beyond the aperture.

In some embodiments, the aperture is designed to encompass an acoustic window of the head of the subject and the probe is configured to emit ultrasound energy into the head of the subject through the acoustic window.

In some embodiments, the probe includes a Transcranial Doppler (TCD) probe.

In some embodiments, the cover includes a plurality of registration marks thereon for facilitating proper alignment of the device with respect to the head of the subject.

In some embodiments, a first one of the registration marks is configured to align with a first anatomical feature of the head of the subject and a second one of the registration marks is configured to align with a second anatomical feature of the head of the subject.

In some embodiments, the first one of the registration marks is a circle for encompassing the first anatomical feature and the second one of the registration marks is an arrow for pointing towards the second anatomical feature.

In some embodiments, the device is first shifted along the head of the subject such that the first one of the registration marks aligns with the first anatomical feature and then the device is rotated such that the second one of the registration marks aligns with the second anatomical feature.

In some embodiments, the headset further includes one or more pads attached to the cover via one or more joints, the one or more pads configured to contact a side of the head of the subject when the headset is placed thereon.

In some embodiments, the one or more joints are configured to stabilize the one or more pads with respect to the head of the subject when the device is shifted and rotated due to pivotal freedom of movement provided by the one or more joints.

In some embodiments, the device is rotated by rotating an entirety of the headset.

In some embodiments, the first anatomical feature is a tragus of the subject and the second anatomical feature is an eye of the subject.

In some embodiments, the device is positioned at a default position within the cover such that the head of the subject is viewable from outside of the cover and beyond the device.

In some embodiments, the headset further includes a mounting plate adapted to receive and hold the device.

In some embodiments, the mounting plate defines an opening adapted to be proximate an area encompassing an acoustic window of the head of the subject such that the area is viewable from outside of the cover.

In some embodiments, the device is configured to slide along the mounting plate such that the device can be aligned with respect to anatomical features of the head of the subject.

In some embodiments, the entire cover is see-through.

In some embodiments, a first portion of the cover is see-through and a second portion of the cover is opaque.

According to various embodiments, there is provide a method of manufacturing a headset for placing on a head of a subject. The method includes providing a device. The method further includes affixing a cover over the device. At least a portion of the cover is made from a see-through material such that a portion of the head of the subject is viewable through the cover.

### BRIEF DESCRIPTION OF THE FIGURES

Features, aspects, and advantages of the present invention will become apparent from the following description and the accompanying example embodiments shown in the drawings, which are briefly described below.

FIG. 1A illustrates a side view of a diagram illustrating the skeletal anatomy of a human head.

FIG. 1B illustrates a side view of a diagram illustrating the muscular anatomy of a human head.

FIG. 2 illustrates a perspective view of a headset positioned on a subject according to various embodiments.

FIG. 3A, FIG. 3B, and FIG. 3C illustrate various views of a headset according to various embodiments.

FIG. 4A, FIG. 4B, and FIG. 4C illustrate various views of the headset shown in FIGS. 3A-3C and a device attached thereto according to various embodiments.

FIG. 5A, FIG. 5B, FIG. 5C, and FIG. 5D illustrate various views of the headset and the device shown in FIGS. 4A-4C and including a cover according to various embodiments.

FIG. 6 illustrates a side view of the device including the cover according to various embodiments.

FIG. 7 illustrates a perspective view of a headset and device including a cover according to various embodiments.

FIG. 8 illustrates a side view of a headset and device including a cover according to various embodiments.

FIG. 9A illustrates a perspective view of a headset and device including a cover according to various embodiments. FIG. 9B illustrates a side view of the headset and the device including the cover shown in FIG. 9A according to various embodiments.

FIG. 10A illustrates a perspective view of the headset and the device including the cover shown in FIGS. 9A-9B positioned on a head of a subject according to various embodiments. FIG. 10B illustrates a top view of the headset and the device including the cover shown in FIG. 10A positioned on the head of the subject according to various embodiments.

FIG. 11 illustrates a Transcranial Doppler system including the headset and device including the cover shown in FIGS. 9A-9B according to various embodiments.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various arrangements, reference is made to the accompanying drawings which form a part hereof and in which are shown, by way of illustration, specific arrangements in which the arrangements may be practiced. It is to be understood that other arrangements may be utilized, and structural changes may be made without departing from the scope of the various arrangements disclosed in the present disclosure.

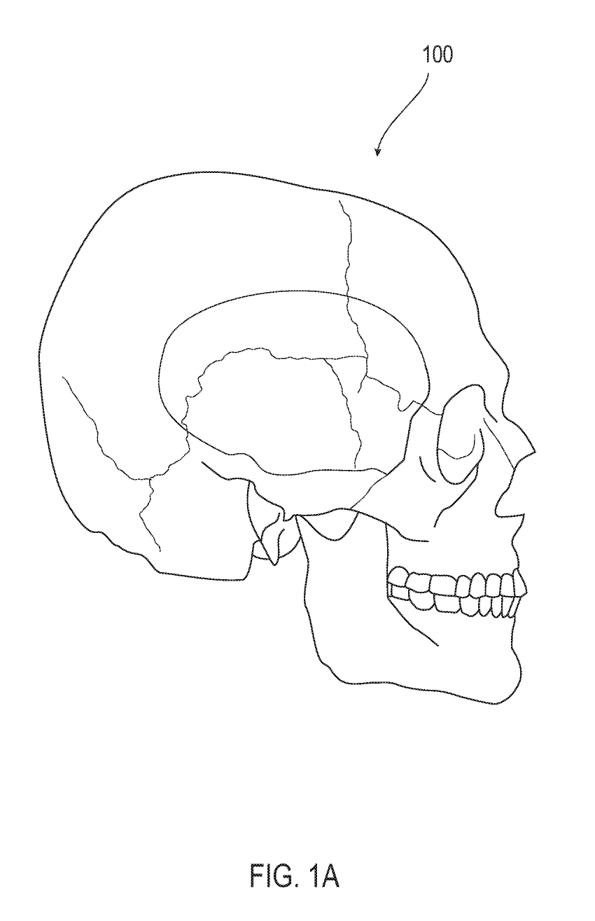

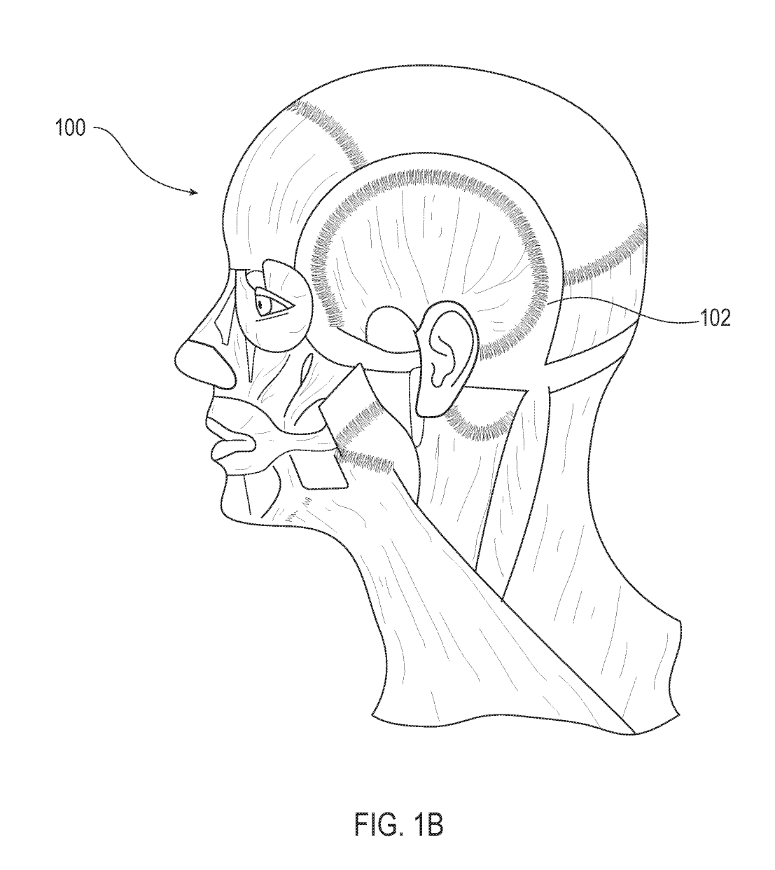

FIG. 1A is a side view of a diagram illustrating the skeletal anatomy of a human head 100. FIG. 1B is a side view diagram illustrating the muscular anatomy of the human head 100.

Referring to FIGS. 1A-1B, the human head 100 includes numerous muscles. However, when a headset including or otherwise supporting a probe is held fast to a portion of the human head 100 that includes muscle tissue, the headset (and therefore the probe) may shift when a user flexes or uses the muscle tissue. As such, affixing the headset to a muscular portion of the head, or strapping the headset around the entire head, may not be desirable when stabilization and alignment of the probe is vital to generating accurate measurements. As such, in some arrangements, a headset is designed to be affixed to a portion of a human's head that contains minimal or no muscle so that a probe connected to the headset is not disturbed or moved during operation.

For example, the human head 100 includes a muscular section referred to as the temporalis, which is a fan-shaped muscle that runs from the side of the skull to the back of the lower jaw and is involved in closing the mouth and chewing. Around the temporalis muscular area, the human head 100 includes a bordering ring section referred to as the temporal fascia 102 (shown in FIG. 1B as surrounding the temporalis region). The temporal fascia 102 is an area that has minimal or no muscle, and may include the user's skin and bone only. The temporal fascia region 102 includes only bone and skin, and begins at the zygomatic process of the frontal bone, continues along the superior and inferior temporal lines on the parietal bone, and further continues around the back of the ear on the mastoid portion (above the mastoid process) of the temporal bone.

According to some arrangements, a headset is held fast to the temporal fascia 102 of a user so that muscular activation or flexing by the user will not disturb the positioning of a probe attached to the headset. Examples of probes suitable for attaching to the disclosed headset are provided in U.S. patent application Ser. No. 15/187,397 and U.S. patent application Ser. No. 15/399,735, the disclosures of both are incorporated herein by reference in their entireties. The temporal fascia 102 provides a stable location for holding a headset to a user's head, as the temporal fascia 102 includes minimal or no muscle tissue. In addition, in some arrangements, because the area of the temporal fascia 102 is primarily a boney structure (e.g., between the superior and inferior temporal lines shown in FIG. 1A), a headset secured to this area provides increased comfort to a user, as the headset applies pressure to bone rather than soft tissue and muscle.

In addition, in some arrangements, a headset can be held fast to the galea aponeurotica region of the head, which is a tough fibrous band that extends over the cranium. The galea aponeurotica also provides a stable location for minimizing movement of the headset and probe during operation. In some arrangements, adjustment of the headset focuses pressure on the galea aponeurotica (e.g., adjustment of the headset downwards into the user's skull). In some arrangements, the headset is held fast to the galea aponeurotica in addition to being held fast to the temporal fascia 102. In other arrangements, the headset is held fast to the galea aponeurotica instead of to the temporal fascia 102. In yet other arrangements, the headset is held fast to the temporal fascia 102, but not to the galea aponeurotica. In further arrangements, a rear strap of the headset is included and tracks, without touching, or contacts the external occipital protuberance on the occipital bone.

Accordingly, in various arrangements, a headset is configured to be held fast to a user's head so as to avoid the muscular portions of the head that can shift the headset during operation. For example, in some arrangements, a headset is configured to avoid being held fast to muscles of a user, such as, but not limited to, temporalis (temporal fossa), orbicularis oculi, frontal belly, occipital belly, facial muscles, and neck muscles (e.g., masseter sternocleidomastoid, trapezius, and capitis).

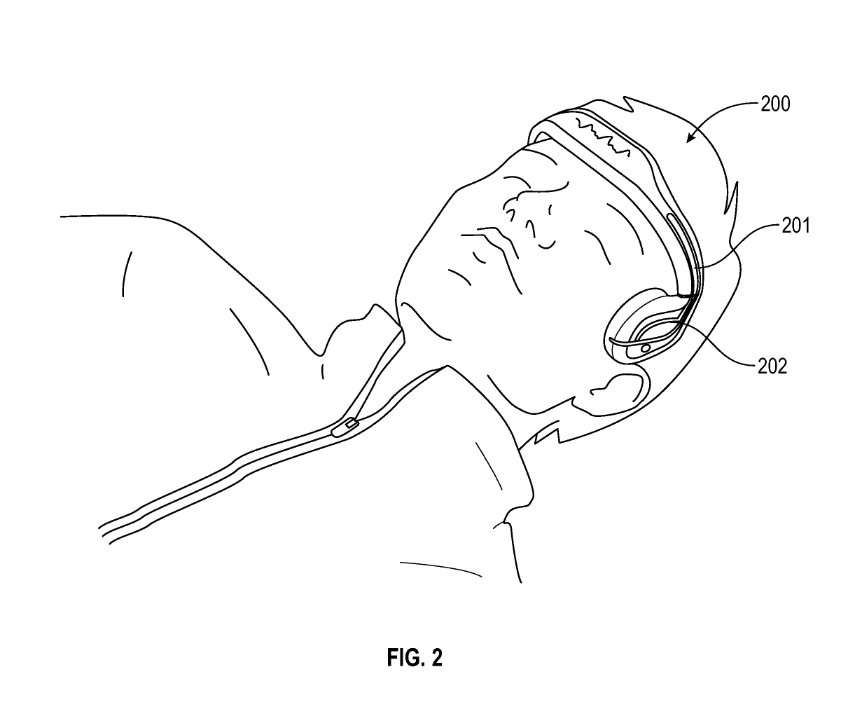

FIG. 2 illustrates a perspective view of a headset 200 positioned on a subject according to various arrangements. In some arrangements, the headset 200 includes a predominantly C-shaped or U-shaped band 201 (e.g., as viewed from an overhead perspective of the headset 200) that is attached or affixed to a subject's head in horizontal or vertical orientations, such as at the front, back, or top of the head. The headset 200 may include a band of another suitable shape in other arrangements. One drawback of traditional headsets is that they contact (e.g., encircle) and tighten around much of the head so that once attached, the headset contacts and is affected (e.g., movable) by numerous muscles (e.g., those shown in FIG. 1B). Accordingly, movement of any one muscle contacted by the headset can result in movement of the entire headset, resulting in poor overall stability. Furthermore, if the headset 200 is attached to a device whose performance is optimized by the headset 200 remaining stable (or at a fixed position) with respect to a user's head (e.g., optical devices, surgical devices, scanning devices, medical diagnostic devices, automated Transcranial Doppler devices, and so on), abrupt movement of the device via the attached headset may compromise the accuracy of readings made by the device during its operation.

As such, in some arrangements, the headset 200 being substantially U-shaped or C-shaped can reduce the number of contact locations between the headset 200 and the human head, and therefore provides less opportunity (e.g., fewer numbers of points of contact) for the headset 200 to shift or move once affixed to the human head. Accordingly, a device attached to the headset 200 may provide more accurate or precise readings during operation. In some arrangements, the headset 200 includes additional straps for securing the headset 200 to a human head.

In various arrangements, the headset 200 is made from any suitable rigid material that maintains its shape. For example, in some arrangements, the headset 200 is made from a material, such as, but not limited to, hard plastic, metals, aluminum, steel, titanium, magnesium, various alloys, rigid plastics, composites, carbon fiber, fiber glass, expanded foam, compression molded foam, stereolithography (SLA) or Fused Deposition Modeling (FDM)-made materials, Reaction Injection Molding (RIM) molding, acrylonitrile butadiene styrene (ABS), thermoplastic olefin (TPO), nylon, polyvinyl chloride (PVC), fiber reinforced resins, or the like.

In some arrangements, further stability is achieved by the headset 200 being designed to squeeze the subject's head, for example, by utilizing padding that is placed at an inner surface of the headset 200 such that the padding is pressed against the head or hair of the subject when the headset 200 is worn. In some arrangements, the headset 200 includes ear hooks or temple tips that are configured to squeeze the bony area (e.g., mastoid process) behind the head.

In some arrangements, the headset 200 (e.g., the band 201) includes a probe unit 202 attached thereto. In some arrangements, the probe unit 202 includes an ultrasound probe that performs ultrasound scans with respect to a subject's head, and the scans include, but are not limited to, Transcranial Doppler (TCD) or Transcranial Color Doppler Sonography (TCCS) with Contrast-Enhanced Transcranial Color Doppler Sonography (CE-TCCS). In some arrangements, the probe unit 202 is configured to be manually operated by an operator. In other arrangements, the probe unit 202 includes a robotic assembly that can move a probe independent of positioning of the headset 200. In some arrangements, the headset 200 is configured to hold the probe unit 202 over and aligned with the general temporal window region of the subject. However, in other arrangements, the headset 200 is configured to hold the probe unit 202 over other regions of the head having different ultrasound windows. In various arrangements, the probe unit 202 is a singular probe, an assembly, or an array of probes. In various arrangements, the probe unit 202 is an electrical unit such as a phased array antenna system capable of beam steering.

One potential problem with comparable headsets including a mechanical assembly placed on the head is that hair may obstruct or become tangled within the mechanical assembly. However, in some arrangements, the headset 200 includes the probe unit 202 that is surrounded by a cover that protects the inner mechanics of the probe unit 202. Further details related to a cover of a device (e.g., the probe unit 202) are discussed below.

In various arrangements, the C-shaped or U-shaped band 201 has a predetermined spring constant so that the headset 200 remains clamped on the subject's head at a desired location. For example, in some arrangements, a default width of the headset 200 is less than a width of a subject's head, but the width of the headset 200 can be elongated by widening it such that the spring constant imparts an inward force on the head once the headset 200 is placed thereon. Accordingly, in various arrangements, the headset 200 is configured as a preset size unique to a subject's head shape and simply slides on to the head such that the spring constant of the headset 200 provides the clamping force. In some arrangements, the spring constant of the headset 200 is adjustable to increase or decrease the distance between the C-shaped or U-shaped band 201 and the head to provide better fit.

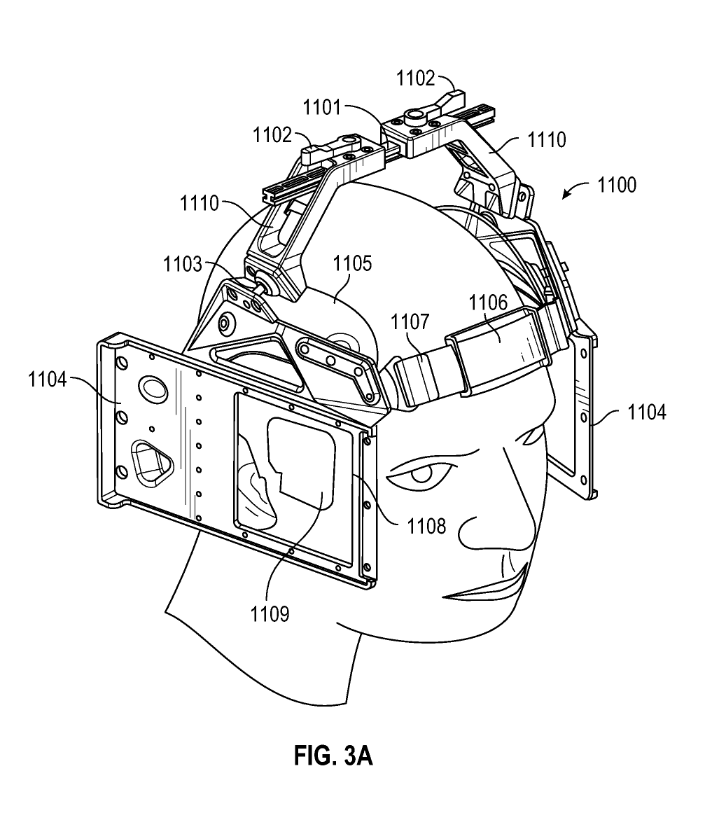

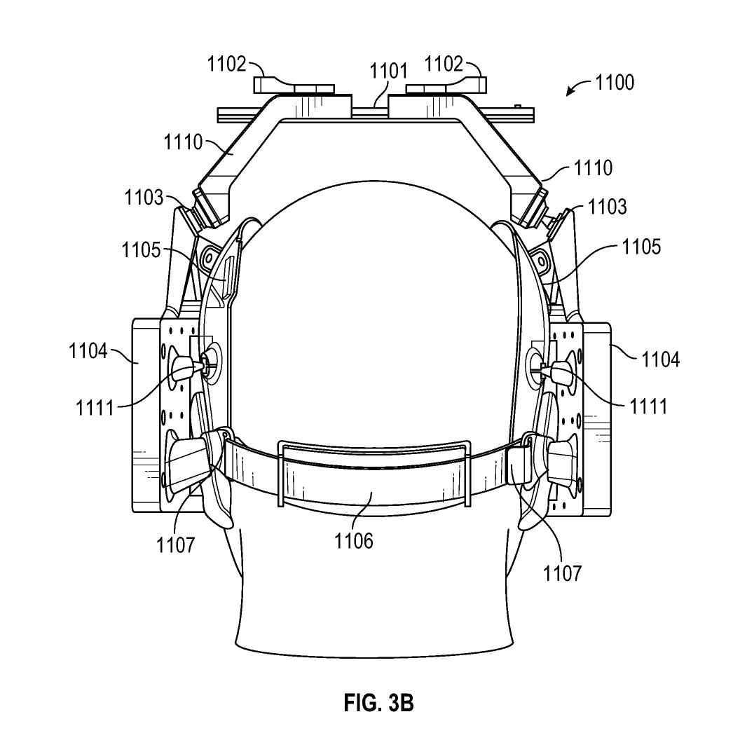

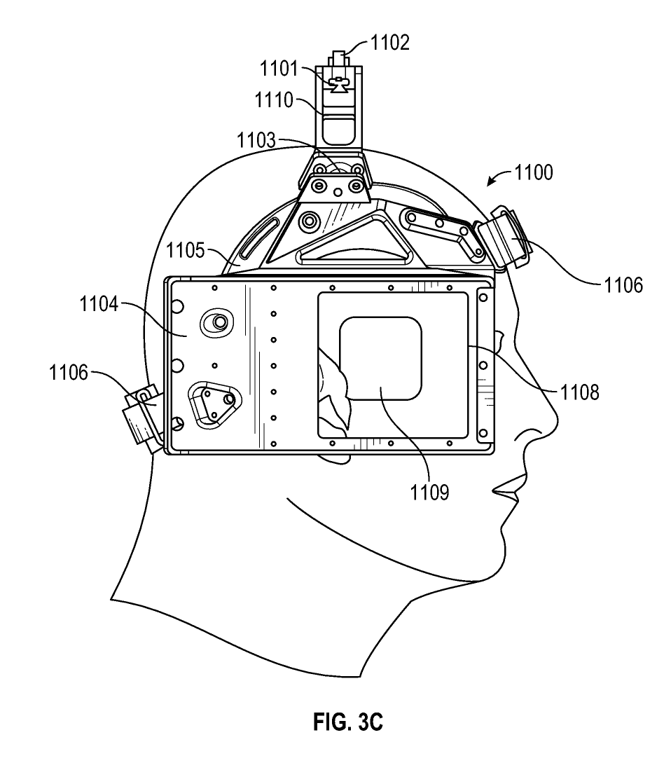

FIG. 3A, FIG. 3B, and FIG. 3C illustrate various views of a headset 1100 according to various arrangements. According to some arrangements, the headset 1100 includes two side arms 1110 connected with a linear slide 1101. In various arrangements, the headset 1100 is made from a rigid material that is configured to maintain its shape. For example, some or all of the headset 1100 is made from any suitable rigid material, such as, but not limited to, hard plastic, metals, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like. In some arrangements, the frame of the headset 1100 is made from any suitably malleable material that allows for flexing, such as, but not limited to, flexible plastics, polyethylene, urethanes, polypropylene, ABS, nylon, fiber-reinforced silicones, structural foams, or the like.

In some arrangements, the headset 1100 includes a mounting plate 1104 for receiving and having attached thereto accessories such as an ultrasound probe, a robotic ultrasound probe, a phased array, and other accessories as described herein. The linear slide 1101 and the side arms 1110 form a rigid structure with minimal flex to serve as a base for the headset 1100. Comparable headsets include flexible materials, but no central rigid region that supports the symmetry of the headset. In some arrangements, the linear slide 1101 is machined to fit into a groove in the side arm 1110. In some arrangements, a locking mechanism 1102 (e.g., a screw, a knob, or the like) is adjusted to tighten against the linear slide 1101 to keep the linear slide 1101 stable and locked in place.

In some arrangements, a pivot point 1103 is adjustable along two degrees of freedom or more and includes a ball pivot, hinge, or other mechanism. In some arrangements, the pivot point 1103 includes a ball pivot capable of three degrees of freedom (e.g., as shown in FIG. 3A). In some arrangements, the pivot point 1103 is operated in conjunction with the linear slide 1101 to move the mounting plate 1104 so that it is aligned with an acoustic window 1109 of a subject's head. Accordingly, in some arrangements, the headset 1100 enables multi-angular, fluid adjustment that allows, for example, tilt along a first axis (e.g., y-axis tilt), tilt along a second axis (e.g., z-axis tilt), rotation along the second axis (e.g., z-axis rotation), rigid clamping along the second axis (e.g., z-axis rigid clamping), rotation along a third axis (e.g., x-axis rotation), and adjustment of a width of the headset 1100 to fit the subject's head and for positioning of the mounting plate 1104.

In some arrangements, a strap 1106 including an integrated pad is affixed across a front of the subject's head and attached to the mounting plate 1104 (e.g., to the two mounting plates 1104 at each side of the headset 1100). In some arrangements, the pad is made from any suitable soft material, such as, but not limited to, closed cell foam, open cell foam, self-skinning open or closed cell foams, cast, aerated, or extruded silicone or urethane, polyurethane gels that are configured to distribute pressure efficiently, or the like. In some arrangements, the pad has any suitable firmness for supporting a head, such as, but not limited to, in a range of about 0.1 pound per square inch (psi) to about 60 psi (e.g., in a range of about 0.1 psi to about 10 psi) or within other suitable ranges of firmness. In some arrangements, the pad has memory for expanding to fit contours of a head. In some arrangements, the pad (e.g., foam) is compressed and expands after a user's head is placed against the pad so that it expands to secure the headset 1100. In some arrangements, the pad is manufactured by any suitable process for affixing the pad within the headset 1100, such as, but not limited to, injection molding, laminating, adhesive mounting (e.g., gluing or bonding), co-molding, co-casting, injection, snapping, by Velcro fastening, by hook and loop fastening, friction fitting, attaching with barbs, using screw bosses, or the like.

In some arrangements, the strap 1106 is tightened or loosened depending on desired fit of the headset 1100 and is held in place with a strap lock 1107. In some arrangements, the strap lock 1107 includes, but is not limited to, Velcro, a ratcheting strap, a hook and loop, or any other suitable locking mechanism. In some arrangements, an additional strap (e.g., similar to the strap lock 1107) is held across the back of the head and also attached to both mounting plates 1104.

In some arrangements, footing 1105 contacts the head. In some arrangements, the footing 1105 is made of foam or any other suitable soft material described in detail above. In some arrangements, the footing 1105 is rigidly mounted to the mounting plate 1104. In other arrangements, the footing 1105 is mounted to the side arm 1110 or to both the mounting plate 1104 and the side arm 1110. In some arrangements, the footing 1105 is mounted on a hinge or pivot to provide further adjustability of the headset 1100. In some arrangements, the footing 1105 is configured to contact portions of a subject's head that include little or no muscle (e.g., the temporal fascia 102) such that muscle movements by the subject will not affect the positioning of the headset 1100.

In some arrangements, the mounting plate 1104 has an opening 1108 for providing access to the acoustic window 1109. In some arrangements, access to the acoustic window 1109 via the opening 1108 enables a probe (e.g., an ultrasound probe) or other measurement device to protrude beyond the headset 1100 and towards the subject's head so that the probe is proximate or contacts the head near or at the acoustic window 1109. In some arrangements, the mounting plate 1104 is positioned so that the opening 1108 is substantially aligned with or encompasses the acoustic window 1109.

In some arrangements, the two side arms 1110 are connected together via the linear slide 1101. The mounting plate 1104 is connected to the side arm 1110 via the pivot point 1103 so as to provide at least one degree of freedom of movement of the mounting plate 1104 with relation to the side arm 1110. In some arrangements, the strap 1106 is positioned to be at the back of the subject's head, and the strap 1106 connects the mounting plates 1104 such that when the strap 1106 is fixed at a desired position the mounting plates 1104 are maintained at a desired position. The combination of the pivot point 1103 and the linear slide 1101 enables additional degrees of freedom so that the mounting plates 1104 can be aligned with suitable anatomical features on the head such as, but not limited to, the acoustic window 1109, nose, eyes, ears, or any other suitable anatomical feature. In some arrangements, one or more of the mounting plates 1104 includes an accessory or device permanently connected thereto. In other arrangements, one or more of the mounting plates 1104 includes an accessory or device releasably connected thereto (e.g., via one or more disconnect mechanisms).

In some arrangements, a joint 1111 is connected between the pad 1105 (that contacts the subject's head) and the mounting plate 1104. The joint 1111 allows the pad 1105 to suitably contact the head, for example, by maximizing the contact area between the pad 1105 and the head. In some arrangements, the pad 1105 and/or the joint 1111 is located on either the side arm 1110 or the mounting plate 1104 depending on the type of accessory or device attached to the mounting plate 1104 and the application thereof.

In some arrangements, the side arm 1110 of the headset 1100 is shaped to hold the linear slide 1101 with a lip, but other shapes or mechanisms (e.g., ball bearings) can be used. In some arrangements, the linear slide 1101 enables independent movement of each side arm 1110 from the other to accommodate the shape of the head. In some arrangements, an additional degree of freedom is provided by adding, to the linear slide 1101, one or more pivot points at each side arm 1110.

In some arrangements, the linear slide 1101 is adjusted to tighten the clamping force exerted by the footings 1105 on the subject's head. In some arrangements, the pivot point 1103 that is connected at the side arms 1110 provides upward and downward freedom of movement along the head by the footings 1105 as the linear slide 1101 is tightened or loosened. For example, in response to tightening of the linear slide 1101, the side arms 1110 are shifted upwards via the pivot points 1103 such that the footings 1105 and the rest of the headset 1100 (e.g., the mounting plate 1104) are also shifted upwards along the subject's head. As such, the height of the headset 1100 can be adjusted with respect to the subject's head. Accordingly, a device (described below) that is attached to the mounting plates 1104 can also be shifted up and down along the subject's head so that an operator can suitably position the device vertically with respect to the subject's head (e.g., so that a probe of the device is properly aligned or registered with respect to the acoustic window 1109 of the subject).

In some arrangements, the joint 1111 further allows the footing 1105 to conform to the subject's head even when the headset 1100 is being adjusted vertically along the head via the linear slide 1101 by providing pivoting freedom of motion at the joint 1111. In some arrangements, there are more than one footings 1105 along one side of the head and each of the footings 1105 are attached to an individual joint (such as but not limited to, the joint 1111). For example, the headset 1100 can include three separate footings 1105 along one side of the head, and each of the footings 1105 is connected to the rest of the headset 1100 via one joint 1111 such that there are three joints 1111 altogether. In some arrangements, one joint 1111 is located towards the front of the headset 1100, a second joint 1111 is located towards the rear of the headset 1100, and a third joint 1111 is located between the first joint 1111 and the second joint 1111 (e.g., at a midway point therebetween).

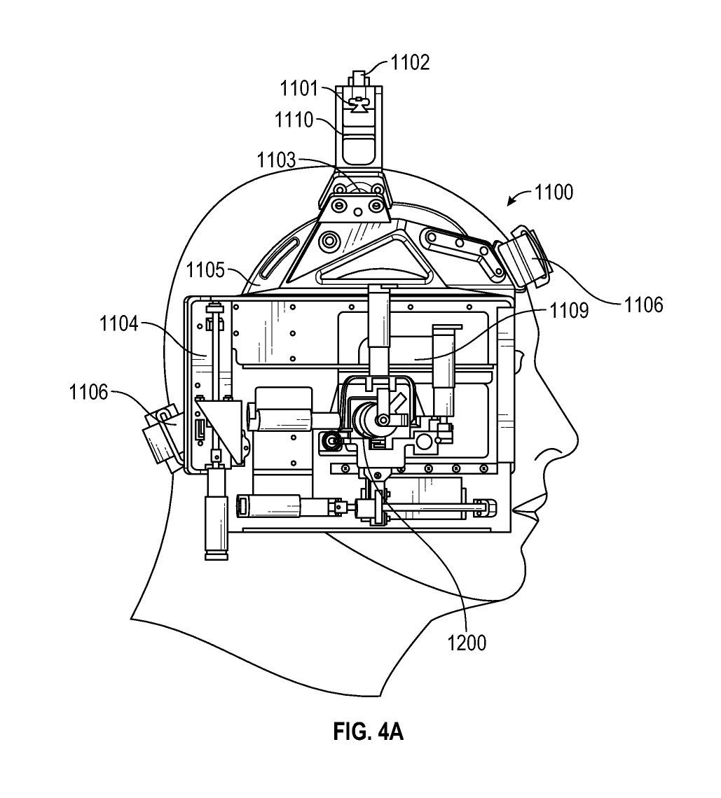

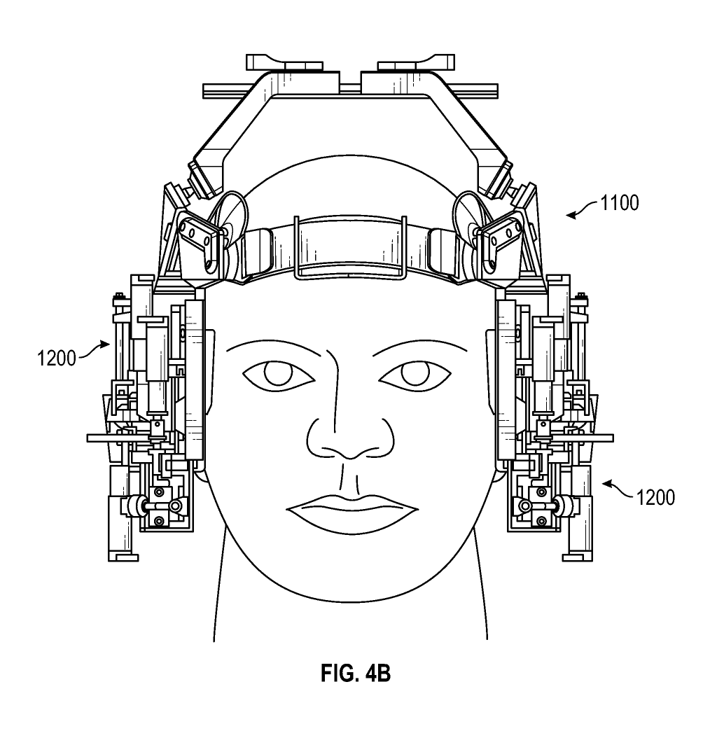

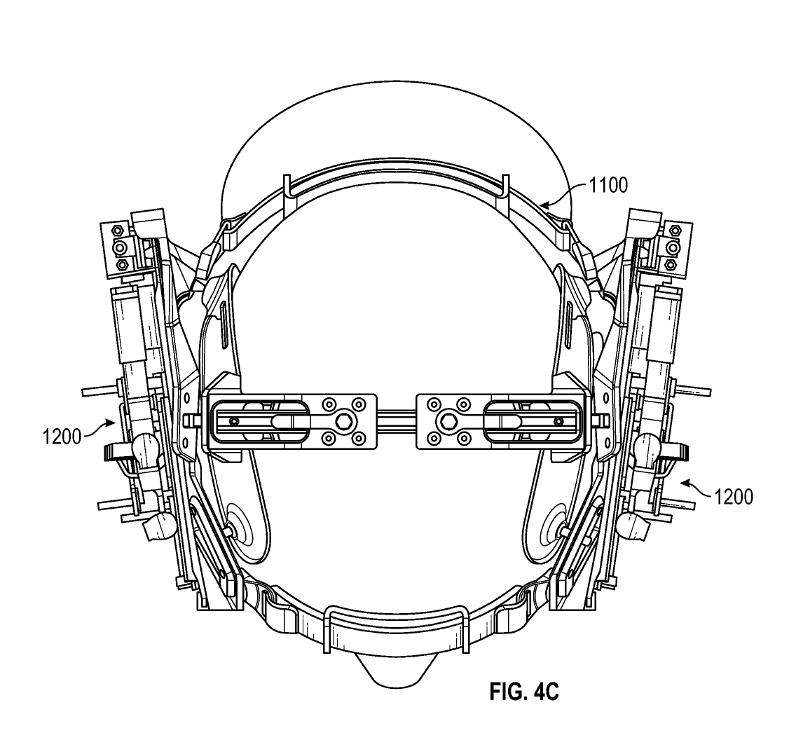

FIG. 4A, FIG. 4B, and FIG. 4C illustrate various views of the headset 1100 shown in FIGS. 3A-3C and a device 1200 attached thereto according to various arrangements. In some arrangements, the device 1200 includes a robotic mechanism and is attached to the mounting plate 1104 or directly to the headset 1100 (or to both). In some arrangements, the device 1200 is configured to move a probe along the anatomy of the subject's head to measure surface or internal anatomy or physiology. In some arrangements, the probe includes an ultrasound probe, and other probes (e.g., near-infrared spectroscopy (NIRS)), a camera, or other accessory can also be attached. In some arrangements, the probe travels around and along the acoustic window 1109 to enable improved transmission of acoustic energy to within the skull.

In some arrangements, the device 1200 is modular and can be attached and detached from the headset 1100 via the mounting plate 1104. In some arrangements, the headset 1100 is used in conjunction with a medical device for use with respect to a user's head (e.g., an ocular monitoring system, a breathing device, a device for monitoring neurological activity, a surgical device, a device for monitoring radioactive traces, or any other device used with respect to a subject's head). In other arrangements, the headset 1100 is used in conjunction with a non-medical device for use with respect to a user's head (e.g., a virtual reality eyepiece).

In some arrangements, the device 1200 includes a probe and robotics for controlling the probe. For example, the robotics are configured to translate the probe along a surface of a head and to move the probe towards and away from the head. In some arrangements, an end of the probe interfaces with the robotics, and the robotics include components, such as, but not limited to, a motor assembly and the like for controlling the probe (e.g., control z-axis pressure, normal alignment, or the like of the probe).

In some arrangements, the probe includes a first end and a second end that is opposite to the first end. In some arrangements, the first end includes a concave surface that is configured to be adjacent to or contact a scanning surface (e.g., a head of a subject). The concave surface is configured with a particular pitch to focus generated energy towards the scanning surface. In some arrangements, the device 1200 is a Transcranial Doppler (TCD) apparatus such that the first end of the probe is configured to be adjacent to or contact and align along a human head (e.g., a side of the human head), and the first end of the probe is configured to provide ultrasound wave emissions from the first end and directed into the human head (e.g., towards the brain). In other arrangements, the probe is configured to emit other types of waves during operation, such as, but not limited to, infrared, x-rays, or the like.

In some arrangements, the second end of the probe is coupled to the robotics. In some arrangements, the second end of the probe includes a threaded section along a portion of the body of the probe, and the second end is configured to be secured in the robotics via the threads (e.g., by being screwed into the robotics). In other arrangements, the probe is secured in the robotics by any other suitable connecting means, such as, but not limited to, welding, adhesive, one or more hooks and latches, one or more separate screws, press fittings, or the like.

In other arrangements, the probe is attached within the headset 1100 without any robotics, such that the probe 131 is configured to be manually operated by an operator while the headset 1100 is positioned on a subject's head. For example, a subject's head can be placed in the headset 1100 and an operator can manually shift and orient the probe while the probe is activated.

Further disclosure regarding probe systems that can be used in conjunction with the headsets described herein can be found in non-provisional patent application Ser. No. 15/399,648, titled ROBOTIC SYSTEMS FOR CONTROL OF AN ULTRASONIC PROBE, and filed on Jan. 5, 2017, which is incorporated herein by reference in its entirety.

In some arrangements, the headset 1100 holds other medical and non-medical devices that are used and stabilized with respect to a subject's head. For example, in some arrangements, an ocular device is a device that is optimized by maintaining positioning and alignment with a user's eyes (e.g., if the ocular device is shifted with respect to a user's eyes, performance of the ocular device may decline). In some arrangements, the ocular device is attached at the headset 1100 so as to cover the eyes of a patient. As an example of a non-medical device use with respect to the headset 1100, in some arrangements, the headset 1100 can be used in connection with the ocular device that is a virtual reality device configured to provide a virtual experience to the subject such that any disturbance of the positioning of the ocular device in front of the user's eyes may cause a degradation in the user's virtual experience.

In some arrangements, the ocular device is a medical device designed to track ocular behavior of a subject (e.g., to diagnose whether the user has experienced a concussion). In other arrangements, the ocular device is an ocular diagnosis or treatment tool for determining or adjusting vision of the user. As an example, the ocular device is a device for correcting imperfect vision of a user (e.g., laser eye surgery). As another example, in some arrangements, the ocular device is an ocular diagnostic tool for determining a vision prescription of a user, presence of one or more eye conditions (e.g., glaucoma, cataracts, ocular hypertension, uveitis, or the like), and so on. In some arrangements, the ocular device is designed to cover and interact with both eyes simultaneously or in sequence. In other arrangements, the ocular device is designed to cover and interact with a single eye (e.g., while the other eye remains uncovered). The ocular device can be provided with any of the headset apparatuses described herein.

In some arrangements, more than one device 1200 is attached to the headset 1100 and aligned with anatomical features. Each of the devices 1200 is attached to a respective mounting plate 1104. In some arrangements, the device 1200 includes the robotics that are configured to move a probe. In some arrangements, the probe includes a camera, NIRS, or an ultrasound emitting device. In some arrangements, the headset 1100 provides the ability to adjust the angle and location of the device 1200. In some arrangements, the acoustic window 1109 is located at the temporal window. In other arrangements, the acoustic window 1109 includes the orbital window, the occipital window and/or mandibular window, and the headset 1100 is correspondingly designed to allow the device 1200 to access these various acoustic windows. For example, instead of the acoustic window 1109 being accessible from the temporal window, the headset 1100 can provide the opening 1108 at the occipital acoustic window for allowing the device 1200 access thereto.

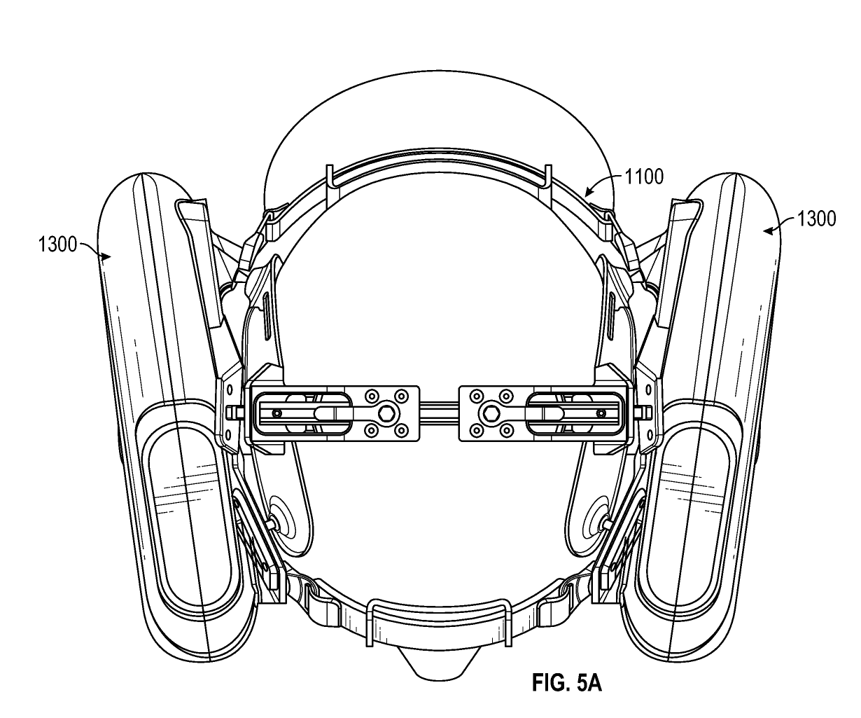

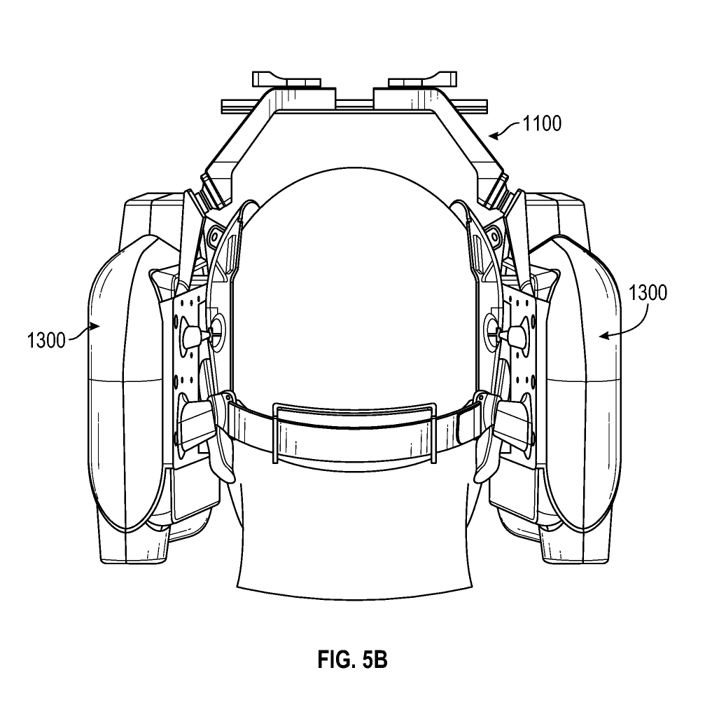

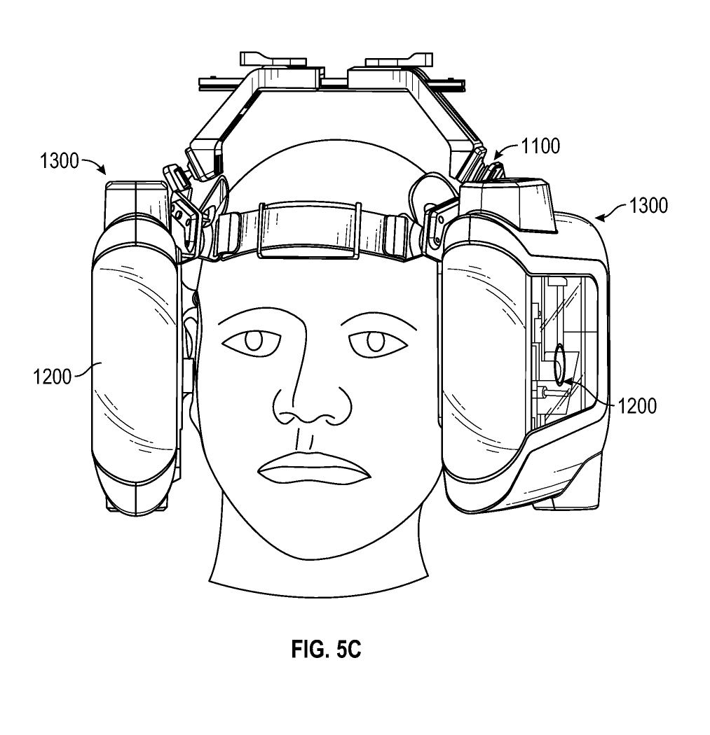

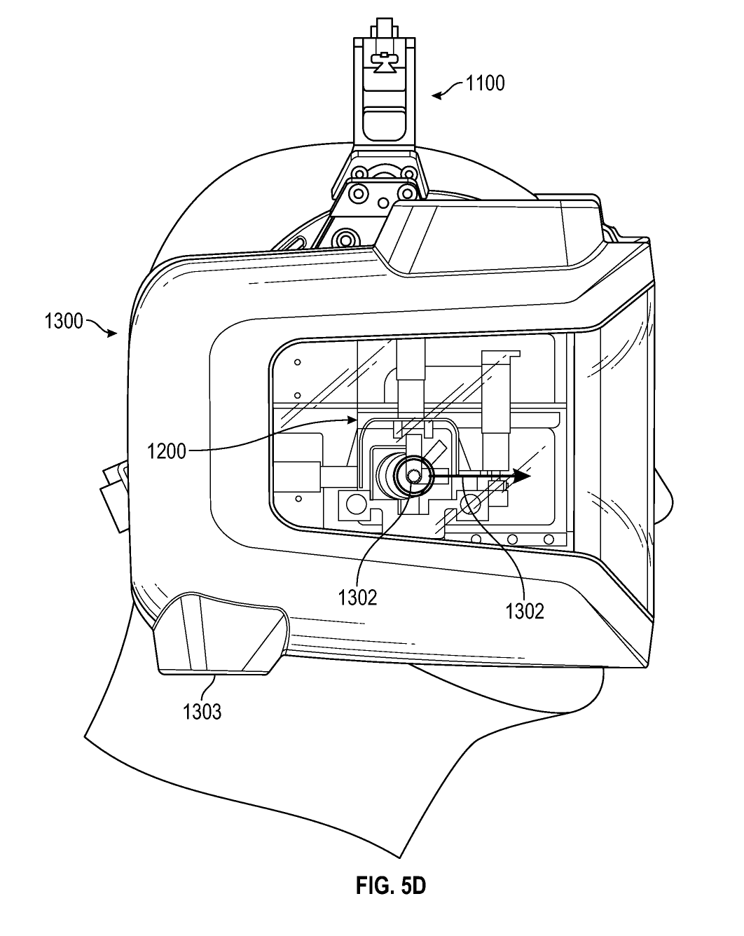

FIG. 5A, FIG. 5B, FIG. 5C, and FIG. 5D illustrate various views of the headset 1100 and the device 1200 shown in FIGS. 4A-4C and including a cover 1300 according to various arrangements. In some arrangements, the cover 1300 is placed over the device 1200 as protection. In some arrangements, the cover 1300 includes two separate portions that are permanently or releasably affixed to each other. In other arrangements, the cover 1300 is made from one piece and includes quick release mechanisms to enable rapid attachment and removal thereof.

In some arrangements, the device 1200 protected by the cover 1300 includes the robotics, which includes, for example, motors, assemblies, and electronics configured to move the probe such that the cover 1300 is used to cover and protect the motors, assemblies, and electronics of the device 1200. Accordingly, the probe of the device 1200 that is attached to the robotics protrudes from the cover 1300 such that the probe is configured to contact or remain proximate to the subject's head. In some arrangements, the cover 1300 is removable from the device 1200 so that it can be serviced without removal from the headset 1100. The cover 1300 is used to protect the device 1200 from hair or other body parts becoming caught therein, as well as to protect the device 1200 from outside damage (e.g., via impact or liquid). In some arrangements, the headset 1100 is utilized for medical applications and the cover 1300 is used to protect the device 1200 from operators inadvertently damaging or moving the probe or robotics while working on the subject. However, in comparable TCD headsets, the entire apparatus and assembly is open to the outside and can be accidentally damaged or inappropriately repositioned.

In some arrangements, the cover 1300 is made from any suitable rigid material, such as, but not limited to, hard plastic, metals, aluminum, steel, titanium, magnesium, various alloys, rigid plastics, composites, carbon fiber, fiber glass, expanded foam, compression molded foam, SLA or FDM-made materials, RIM molding, ABS, TPO, nylon, PVC, fiber reinforced resins, or the like. In some arrangements, the cover 1300 is partially made from see-through material so that the operator can see the assembly and human head from outside of the headset 1100 (e.g., such that other portions of the headset 1100 are opaque). For example, when aligning a probe with the acoustic window 1109, the see-through material enables the operator to see the components of the device 1200 and the human head so that desired alignment can be achieved while adjusting the headset 1100. Furthermore, by providing a view of the device 1200 (e.g., the probe) and the subject's head while the headset 1100 is placed thereon, an operator can properly register the device 1200 with respect to anatomical features of the subject to optimize performance of the device 1200 during operation.

In some arrangements, the cover 1300 includes an opening therein, at the side of the cover facing a subject's head, so that the probe can protrude and make contact with the head. Said opening allows free movement of the probe along the subject's head. In some arrangements, more than one cover 1300 is used depending on the number of devices 1200 (e.g., each of the two devices 1200 is protected with a cover 1300).

In some arrangements, the cover 1300 includes registration marks 1302 that are used by an operator to align the headset 1100 and the device 1200 with anatomical features of the subject's head, such as, but not limited to, ears, eyes, nose, and so on. In particular arrangements, the registration marks 1302 are used to align the device 1200 enclosed by the cover 1300 with respect to desired anatomical features of the subject's head. In some arrangements, the registration marks 1302 include a circle and/or a straight line and an arrow, but other suitable shapes for alignment purposes can be used. In further arrangements, the registration marks 1302 include lights (e.g., light emitting diodes) or other indicators within the cover 1300 to illuminate and aid with registration of the device 1200.

In some arrangements, a first registration mark of the registration marks 1302 (e.g., the circle) is configured to be aligned with a first anatomical feature of a subject's head (e.g., an ear of the subject, for example, the tragus of the subject). A second registration mark (e.g., the line with an arrow at the end) can then be utilized to properly orient the device 1200 by adjusting (e.g., rotating) it such that the arrow points towards a second anatomical feature of the subject's head (e.g., an eye of the subject, for example, a corner of the eye of the subject). In other arrangements, the first anatomical feature and the second anatomical feature include any suitable feature for properly aligning and orienting the device 1200, such as, but not limited to, other portions of the ear, the cheek, the nose, and so on.

In some arrangements, the device 1200 and the cover 1300 are incorporated into the headset 1100 such that the registration marks 1302 can overlap the relevant anatomical features of the subject's head. For example, the design of the device 1200 (e.g., the robotics) can define one or more openings therein so that the subject's head is visible through the see-through portions of the cover 1300. In some arrangements, the robotics of the device 1200 are configured to shift to a default position within the cover 1300 to clear the view for an operator to align the registration marks with anatomical features of the subject's head.

As an example of registration of the device 1200 with respect to the subject's head, an operator can use the see-through portion of the cover 1300 to view the subject's head and align the circle registration mark 1302 with a first anatomical feature of the subject's head (e.g., the subject's tragus), for example, such that the circle registration mark 1302 encompasses the first anatomical feature. In some arrangements, the operator initially adjusts the vertical position of the device 1200 with respect to the subject's head via the linear slide 1101 such that the device 1200 is more aligned with the acoustic window 1109 of the subject. Once the circle registration mark 1302 is aligned with the first anatomical feature of the subject's head, the cover 1300 and the device 1200 are rotated so that the arrow registration mark 1302 is pointing towards a second anatomical feature of the subject's head (e.g., the corner of the subject's eye). In some arrangements, once the circle registration mark 1302 is properly aligned, the arrow registration mark 1302 is rotated into position by rotating the entire headset 1100 into the proper orientation. For example, the joints 1111 allow the frame of the headset 1100 to rotate while maintaining the footings 1105 in place and contacting the subject's head for stability.

In other arrangements, the circle registration mark 1302 is any suitable size or shape to encompass the first anatomical feature. Similarly, the arrow registration mark 1302 is long enough to suitably allow an operator to align the arrow registration mark 1302 with the second anatomical feature. In some arrangements, the registration marks 1302 are spaced apart from each other at a suitable distance far enough to allow an operator to properly register the device 1200. In other arrangements, the registration marks 1302 include a single line for aligning between the first anatomical feature and the second anatomical feature. In some arrangements, the registration marks 1302 include two distinct shapes that are for overlapping with two separate anatomical features of the subject. In some arrangements, the cover 1300 includes a single registration mark 1302 for aligning with a single anatomical feature of the subject. In various arrangements, regardless of the shape or form of the registration marks 1302, the registration marks 1302 are located at any suitable position on the cover 1300 (e.g., the see-through portion of the cover 1300) for facilitating registration of the device 1200. In particular arrangements, the registration marks 1302 are located along a same horizontal plane, on different planes (e.g., at opposite corners of the cover 1300), and so on.

In some arrangements, the cover 1300 includes a port 1303 so that cables can be connected to the device 1200 therein. In some arrangements, the cables are used to supply power, control signals, or otherwise communicate with the device 1200 within the cover 1300. In other arrangements, the device 1200 is fully self-contained and automated (e.g., by using battery power) and/or wireless signals are delivered to the device 1200 from the outside (and wireless signals are received outside from the device 1200). In some arrangements, cables are used to control and power the device 1200 within the headset 1100. In other arrangements, control is completed using a wireless connection. Additionally, in some arrangements, the device 1200 includes a battery such that the device 1200 is fully contained and does not use any wires.

In some arrangements, the cover 1300 is made from a clear material such as plastic, but any other suitable clear material may be used. The clear material enables the operator to place the headset 1100 on a subject while simultaneously visually inspecting the device 1200 within the cover 1300 or the anatomy of the human head.

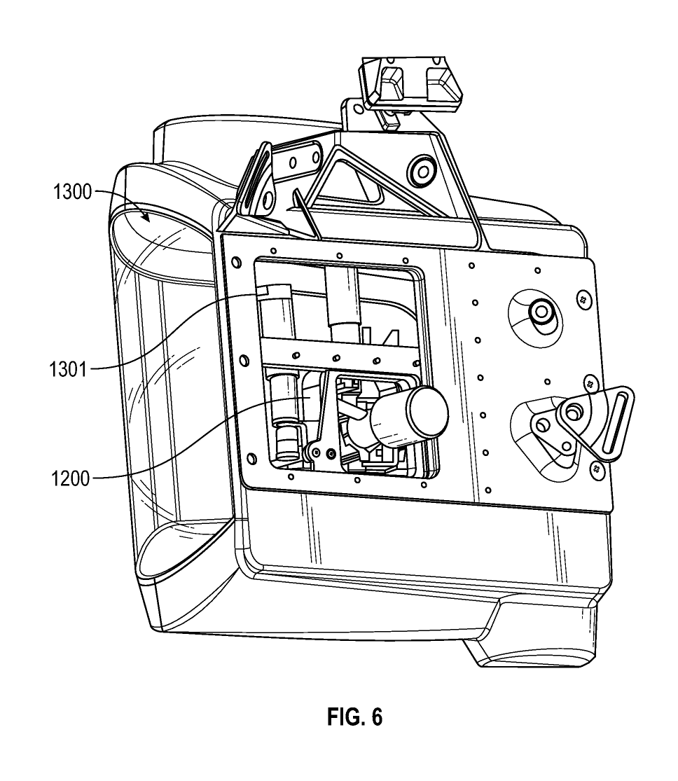

FIG. 6 illustrates a side view of the device 1200 including the cover 1300 according to various arrangements. In some arrangements, the cover 1300 defines an aperture 1301 that allows the probe to protrude therefrom to outside of the cover 1300 such that the probe can contact or be proximate a subject's head distal from the robotic mechanism. In some arrangements, the cover 1300 has a flexible baffle or skirt that covers the aperture 1301. The baffle is configured to protect against particles, liquid, gel, and the like from entering into the robotic mechanism of the device 1200 via the aperture 1301. In some arrangements, the baffle defines an opening through which the probe protrudes.

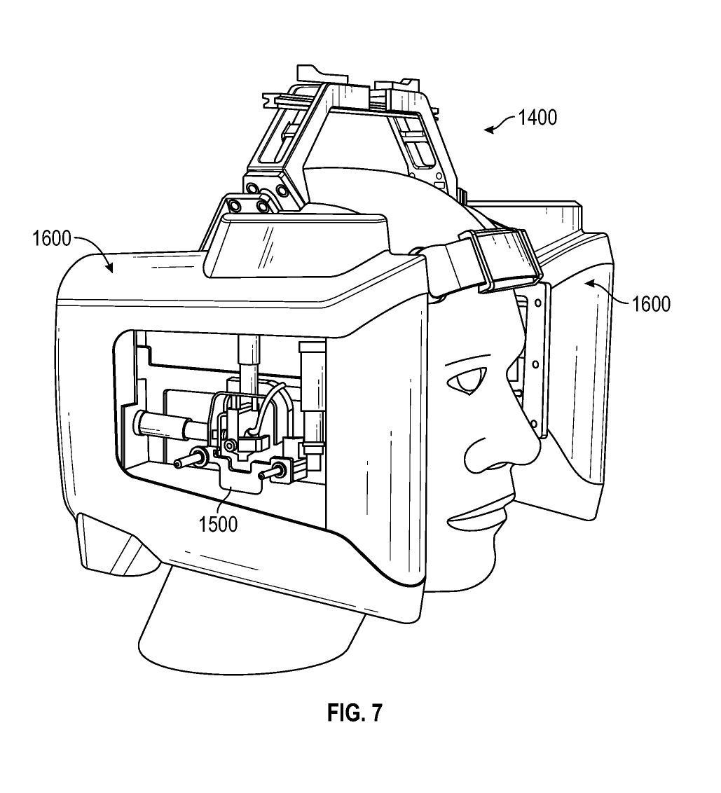

FIG. 7 illustrates a perspective view of a headset 1400 and device 1500 including a cover 1600 according to various arrangements. In some arrangements, the cover 1600 is a partially see-through cover. As such, the device 1500 is viewable so that its location (e.g., the location of the probe) is readily ascertainable by an operator. Additionally, the subject's head can also be seen from outside of the cover 1600 so that the device 1500 (e.g., the probe) can be properly aligned to desired locations on the subject's head. In some arrangements, the desired location is an acoustic window (e.g., the temporal acoustic window) and the robotic mechanism of the device 1500 controls the positioning of an ultrasound probe or phased array about this acoustic window.

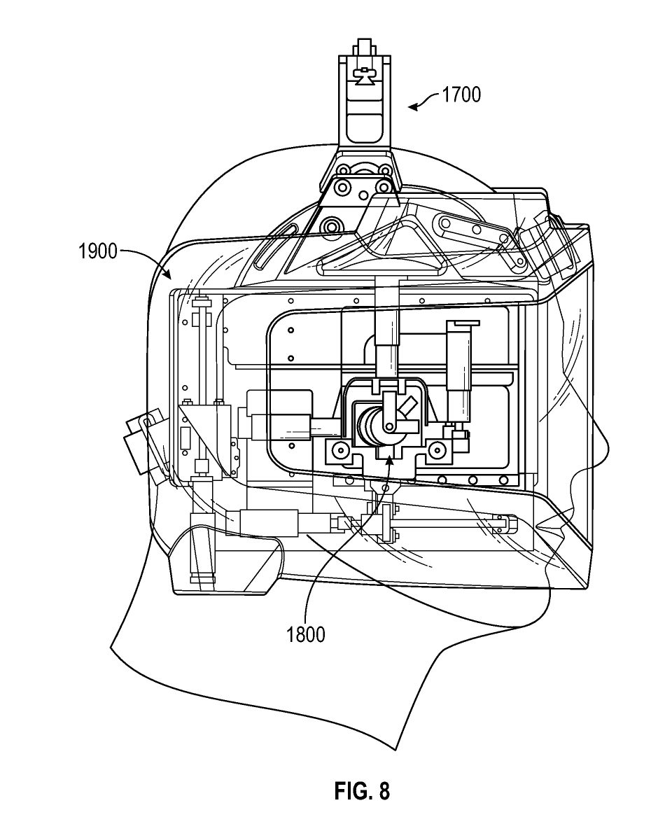

FIG. 8 illustrates a side view of a headset 1700 and device 1800 including a cover 1900 according to various arrangements. In some arrangements, the cover 1900 is mostly made from clear material (e.g., plastic), but other suitable materials or mix of materials can be used. Clear materials allow for an operator to view the robotic mechanism and probe of the device 1800, allowing for easier alignment or registration of the device 1800 with respect to a subject's head. Although FIG. 8 illustrates an example of a robotic mechanism that is used to move an ultrasound probe around the temporal window, other assemblies or mechanical units can be used in connection with the headset 1700.

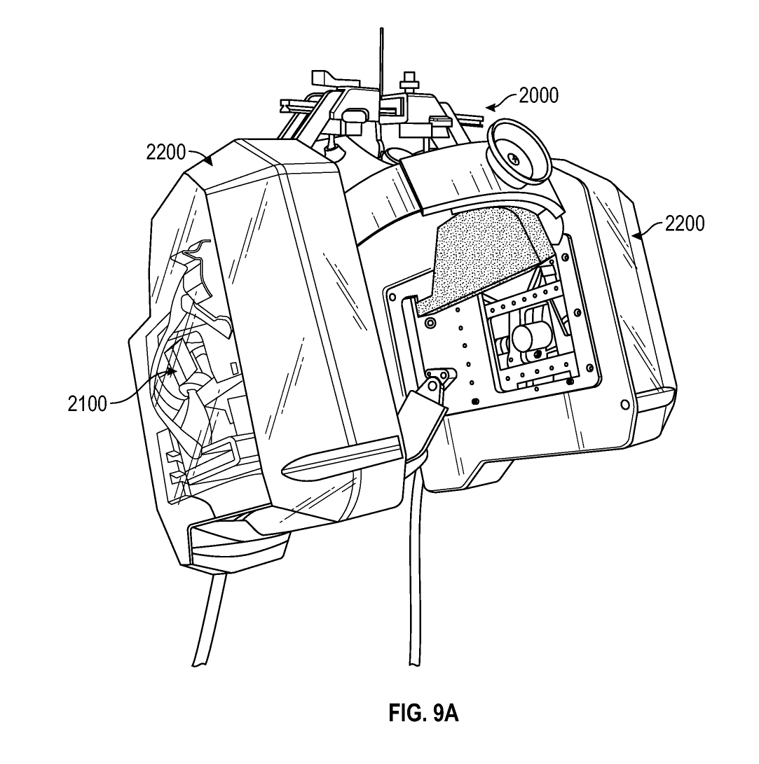

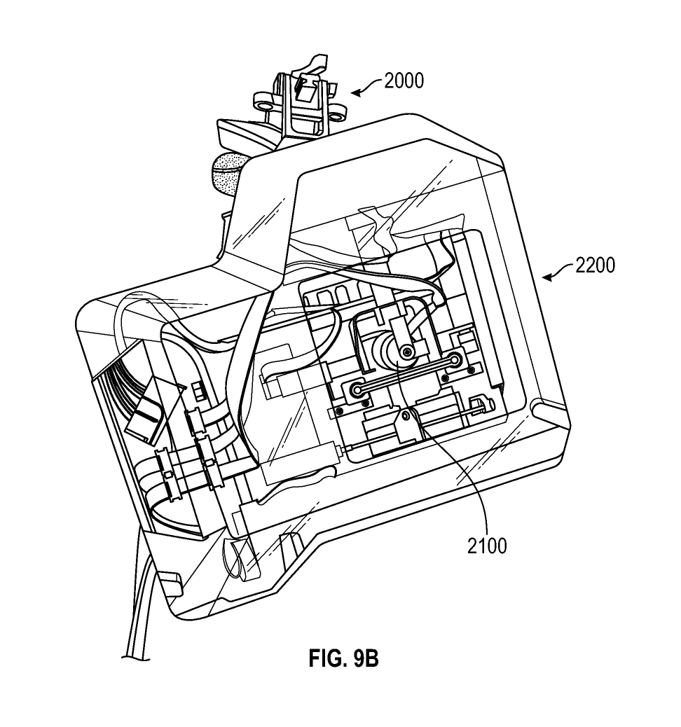

FIG. 9A illustrates a perspective view of a headset 2000 and device 2100 including a cover 2200 according to various arrangements. FIG. 9B illustrates a side view of the headset 2000 and the device 2100 including the cover 2200 shown in FIG. 9A according to various arrangements. In some arrangements, the cover 2200 is substantially made from see-through plastic such that an operator can see the device 2100 and a subject's head on which the headset 2000 is placed.

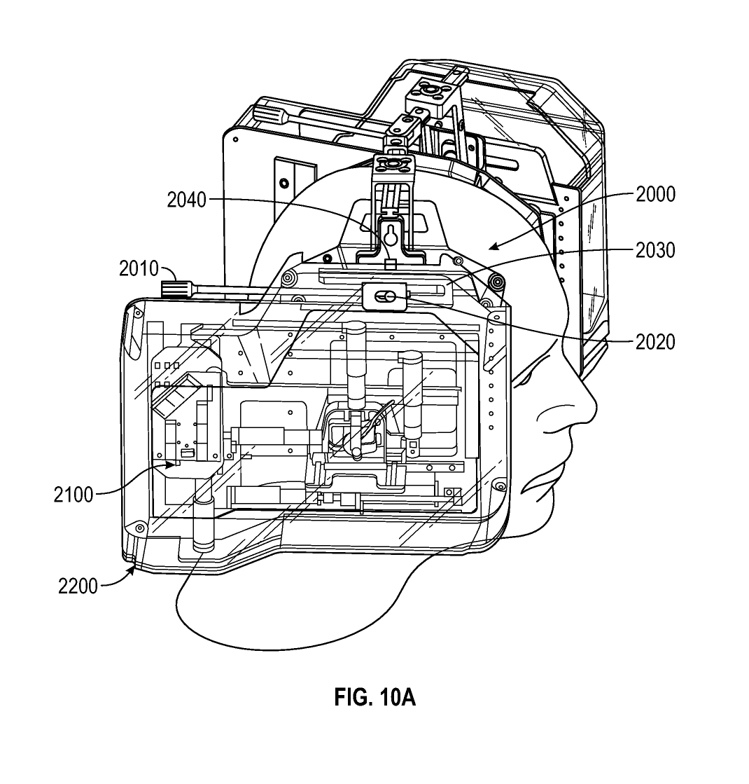

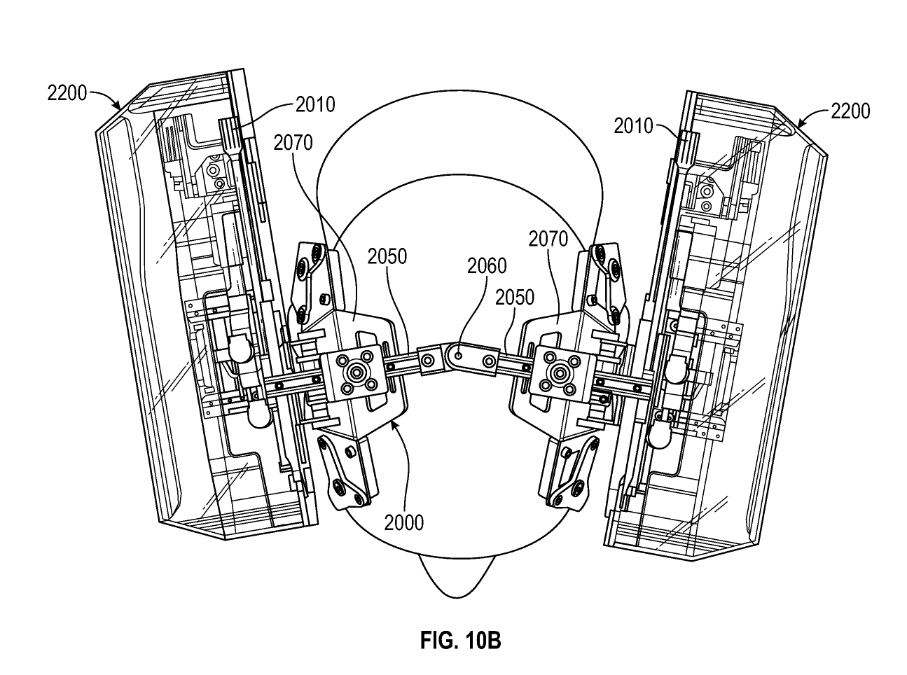

FIG. 10A illustrates a perspective view of the headset 2000 and the device 2100 including the cover 2200 shown in FIGS. 9A-9B positioned on a head of a subject according to various arrangements. FIG. 10B illustrates a top view of the headset 2000 and the device 2100 including the cover 2200 shown in FIG. 10A positioned on the head of the subject according to various arrangements.

The headset 2000 includes a dial 2010, a cylinder 2020, a horizontal rail 2030, and a vertical rail 2040. In some arrangements, the dial 2010 is configured to tighten and lock the device 2100 and cover 2200 in place. The dial 2010 is further configured to loosen so as to allow freedom of movement for positioning of the device 2100 and cover 2200, and once the device 2100 is suitably positioned with respect to the subject's head, the dial 2010 can be tightened to lock the position of the device 2100. For example, when loosened, the cover 2200 containing the device 2100 therein is capable of horizontal adjustment by sliding along the horizontal rail 2030. In addition, the cover 2200 containing the device 2100 is capable of vertical adjustment by sliding along the vertical rail 2040. Furthermore, the cover 2200 containing the device 2100 is capable of rotational adjustment by rotating along an axis defined by the cylinder 2020. All three axes of movement (e.g., horizontal, vertical, and rotation) are locked by tightening of the dial 2010. Accordingly, an operator of the headset 2000 can register the device 2100 with respect to anatomical features of a subject by actuation of the dial 2010 and movement of the device 2100 along multiple axes of motion of the cover 2200.

Furthermore, in some arrangements, the headset 2000 includes a top bar 2050, a knob 2060, and a footing 2070. In some arrangements, the footing 2070 is designed to conform to the shape of the head of the subject so as to provide a comfortable and snug fit of the headset 2000. The top bar 2050 is connected to the footing 2070 at a first end of the top bar 2050 and to the knob 2060 at a second end of the top bar 2050. Upon loosening of the knob 2060, the top bar 2050 is capable of rotation about an axis defined by the knob 2060 such that the footing 2070 can be adjust inwards (e.g., towards the subject's head) and outwards (e.g., away from the subject's head). Upon tightening of the knob 2060, the top bar 2050 is rotationally locked in place such that the footing is also locked in place. Accordingly, the footing 2070 can be adjustable so as to accommodate different sized heads.

In some arrangements, an operator of the headset 2000 utilizes the adjustment mechanisms to register the device 2100 with respect to the head of the subject. For example, in some arrangements, the operator first shifts the cover 2200 including the device 2100 therein such that a probe of the device 2100 is aligned with a first anatomical feature of the subject (e.g., the tragus of the subject or any other suitable feature of the subject), and the device 2100 can input and store its position (e.g., a headset control box 2300, as described below, can store the position of the headset 2000). Then, the operator can physically shift the cover 2200 including the device 2100 using the adjustment mechanisms of the headset 2000 to position the probe of the device 2100 at a second anatomical feature of the subject (e.g., the corner of the eye of the subject or any other suitable feature of the subject), and the device 2100 can input and store its position (e.g., the headset control box 2300, as described below, can store the position of the headset 2000). Accordingly, by storing the first position of the device 2100 (e.g., aligned with the first anatomical feature) and the second position of the device 2100 (e.g., aligned with the second anatomical feature), the device 2100 determines its boundaries and paths of travel with respect to the head of the subject during operation thereof.

In some arrangements, an operator of the headset 2000 measures the distance between the first anatomical feature and the second anatomical feature and inputs that measurement as a parameter of registration of the device 2100 (e.g., inputs that information into the headset control box 2300). In some arrangements, the device 2100 is registered by storing the position of the device 2100 with respect to the first anatomical feature, but not the second anatomical feature, or vice versa. In some arrangements, the operator aligns the probe of the device 2100 for registration (e.g., by storing positions of the device 2100 with respect to anatomical features of the subject's head, as described above) by viewing the location of the probe of the device 2100 with respect to the head of the subject through the see-through cover 2200 and through openings defined by the device 2100 that allow a view of the subject's head from outside of the headset 2000.

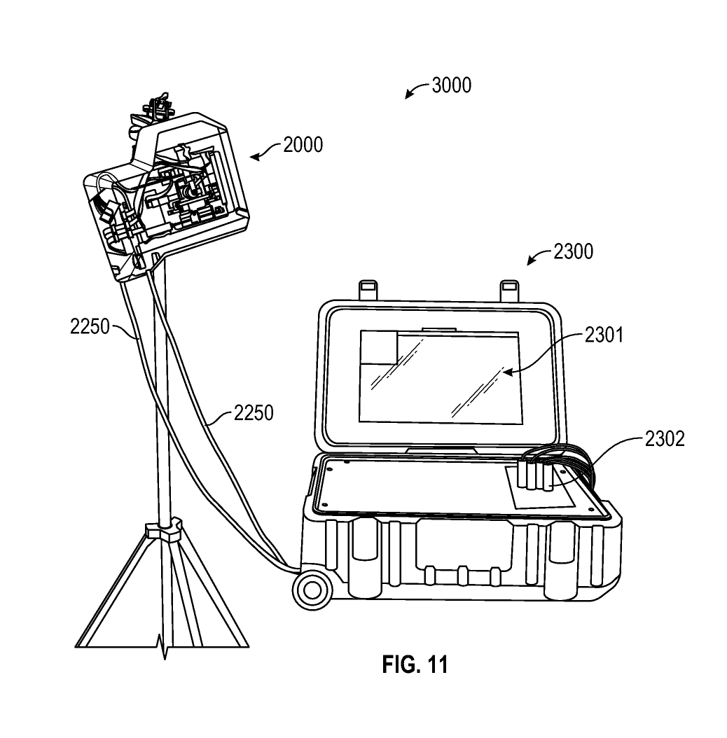

FIG. 11 illustrates a Transcranial Doppler system 3000 including the headset 2000 and device 2100 including the cover 2200 shown in FIGS. 9A-9B according to various arrangements. In some arrangements, the system 3000 includes a headset control box 2300 according to various arrangements. In some arrangements, the headset control box 2300 includes a screen 2301 for viewing readouts or other results (e.g., of the mechanical positioning of the probe of the device 2100 located within the headset 2000 and measurements recorded by device 2100). In some arrangements, the screen 2301 is an independent monitor or a complete computer such as a tablet computer. In some arrangements, the tablet computer controls the device 2100 and connects wirelessly with other computers. However, in other arrangements, other suitable computers or dedicated electronics are used as the headset control box 2300 (e.g., a laptop, desktop, other mobile device, and so on). In some arrangements, the screen 2301 includes a touch screen that enables an operator to control the device 2100. In some arrangements, the headset control box 2300 includes wheels for physical mobility of the headset control box 2300, and such that portability of the entire TCD system 3000 is achieved. In some arrangements, the headset control box 2300 is housed directly in a protective case.

In some arrangements, the headset control box 2300 is configured to control an ultrasound probe of the device 2100 that performs ultrasound scans within a subject's head, such as, but not limited to, TCD, TCCS with CE-TCCS, and so on. In some arrangements, the device 2100 includes a robotic mechanism configured to move the probe independent of the positioning of the headset 2000.

In some arrangements, the headset control box 2300 includes a combination of motor encoders, digital signal processing elements of motor position, a power supply unit, Universal Serial Bus (USB) connectors, an application layer including a touch screen interface for controlling movement of a robot, signal intensity heat maps, batteries, wire connectors, and so on.

In some arrangements, the screen 2301 is configured to visualize recordings as well as position information of the headset 2000 or probes located in the headset 2000. In some arrangements, the headset 2000 is connected to control cables 2250 that are connected to controller connectors 2302 at the headset control box 2300. However, in other arrangements, wireless signals are used to control operation of the device 2100 from the headset control box 2300.

Although headsets disclosed herein are depicted as headsets to be worn while a subject is standing or in a seated position, headsets according to various arrangements can also be designed for subjects laying down (e.g., supine). For example, headsets according to some arrangements are designed to receive a subject's head that is in a supine position such that one or more devices are located at opposite temporal windows of the subject for scanning. In some arrangements, devices attached to a supine headset are protected by covers and the covers have registration marks for aligning with anatomical features of a subject, as described herein.

The above used terms, including “held fast,” “mount,” “attached,” “coupled,” “affixed,” “connected,” “secured,” and the like are used interchangeably. In addition, while certain arrangements have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A headset for placing on a head of a subject comprising:
a device; and
a cover over the device, wherein at least a portion of the cover is made from a see-through material such that a portion of the head of the subject is viewable through the cover.

2. The headset of claim 1, wherein the device includes robotics and a probe attached to the robotics.

3. The headset of claim 2, wherein the cover includes an aperture and the probe protrudes beyond the aperture.

4. the headset of claim 3, wherein the aperture is designed to encompass an acoustic window of the head of the subject and the probe is configured to emit ultrasound energy into the head of the subject through the acoustic window.

5. The headset of claim 2, wherein the probe includes a Transcranial Doppler (TCD) probe.

6. The headset of claim 1, wherein the cover includes a plurality of registration marks thereon for facilitating proper alignment of the device with respect to the head of the subject.

7. The headset of claim 6, wherein a first one of the registration marks is configured to align with a first anatomical feature of the head of the subject and a second one of the registration marks is configured to align with a second anatomical feature of the head of the subject.

8. The headset of claim 7, wherein the first one of the registration marks is a circle for encompassing the first anatomical feature and the second one of the registration marks is an arrow for pointing towards the second anatomical feature.

9. The headset of claim 7, wherein the device is first shifted along the head of the subject such that the first one of the registration marks aligns with the first anatomical feature and then the device is rotated such that the second one of the registration marks aligns with the second anatomical feature.

10. The headset of claim 9, further comprising one or more pads attached to the cover via one or more joints, the one or more pads configured to contact a side of the head of the subject when the headset is placed thereon.

11. The headset of claim 10, wherein the one or more joints are configured to stabilize the one or more pads with respect to the head of the subject when the device is shifted and rotated due to pivotal freedom of movement provided by the one or more joints.

12. The headset of claim 10, wherein the device is rotated by rotating an entirety of the headset.

13. The headset of claim 7, wherein the first anatomical feature is a tragus of the subject and the second anatomical feature is an eye of the subject.

14. The headset of claim 1, wherein the device is positioned at a default position within the cover such that the head of the subject is viewable from outside of the cover and beyond the device.

15. The headset of claim 1, further comprising a mounting plate adapted to receive and hold the device.

16. The headset of claim 15, wherein the mounting plate defines an opening adapted to be proximate an area encompassing an acoustic window of the head of the subject such that the area is viewable from outside of the cover.

17. The headset of claim 15, wherein the device is configured to slide along the mounting plate such that the device can be aligned with respect to anatomical features of the head of the subject.

18. The headset of claim 1, wherein a first portion of the cover is see-through and a second portion of the cover is opaque.

19. The headset of claim 1, wherein the headset is designed to receive the head of the subject that is in a supine position.

20. A method of manufacturing a headset for placing on a head of a subject comprising:
providing a device; and
affixing a cover over the device, wherein at least a portion of the cover is made from a see-through material such that a portion of the head of the subject is viewable through the cover.
